(19) United States
(12) Patent Application Publication
(10) Pub. No.: US 2008/0254511 A1
(43) Pub. Date: Oct. 16, 2008

(54) PROCESS FOR THE FERMENTATIVE PRODUCTION OF PROTEINS

(75) Inventors: Tobias Dassler, Munich (DE); Guenter Wich, Munich (DE); Gerhard Schmid, Gauting (DE)

Correspondence Address: BROOKS KUSHMAN P.C., 1000 TOWN CENTER, TWENTY-SECOND FLOOR, SOUTHFIELD, MI, 48075, US

(73) Assignee: WACKER CHEMIE AG, Munich (DE)

(21) Appl. No.: 11/859,616

(22) Filed: Sep. 21, 2007

(30) Foreign Application Priority Data
Sep. 22, 2006 (EP) 06 121 090.2

(51) Int. Cl. C12P 21/00

(52) U.S. Cl. 435/69.4; 435/69.1; 435/69.6

(57) ABSTRACT

The present invention relates to a process for producing a heterologous protein by means of an E. coli strain in a fermentation medium. The process comprises fermenting an E. coli strain in a fermentation medium. The E. coli strain has a mutation in the lpp gene or in the promoter region of the lpp gene, and contains a gene coding for a heterologous protein which is functionally linked to a signal sequence coding for a signal peptide. The fermentation medium includes Ca²⁺ ions in a concentration above 4 mg/l or Mg²⁺ ions in a concentration above 48 mg/l. The E. coli strain secretes the heterologous protein into the fermentation medium. The protein is removed from the fermentation medium.

### BACKGROUND OF THE INVENTION

1. Field of the Invention

The present invention relates to a process for the fermentative production of heterologous proteins by using an Escherichia coli strain having a lipoprotein mutation.

2. Background Art

The market for recombinant protein pharmaceuticals (pharmaceutical proteins/biologics) has grown greatly in recent years. Particularly important protein pharmaceuticals are eukaryotic proteins, especially mammalian proteins and human proteins. Examples of important pharmaceutical proteins are cytokines, growth factors, protein kinase, protein hormones and peptide hormones, and antibodies and antibody fragments. Because the production costs for pharmaceutical proteins are still very high there is a continuous search for more efficient and more cost-effective processes and systems for producing them.

Recombinant proteins are generally produced either in mammalian cell cultures or in microbial systems. Microbial systems have an advantage over mammalian cell cultures in that recombinant proteins can be produced in a shorter time and at lower cost. Bacteria are therefore particularly suitable for producing recombinant proteins. The organism most frequently used at present for producing recombinant proteins is the Gram-negative enterobacterium Escherichia coli, because its genetics and physiology have been very well investigated, the generation time is short and manipulation is easy. Recombinant proteins can normally be produced in E. coli in various ways:

1. intracellular production as soluble protein;
2. intracellular production as inclusion bodies;
3. secretion into the periplasm.

Accumulation of the target protein in the periplasm has various advantages over intracellular production: 1) the N-terminal amino acid residue of the secreted target protein need not necessarily be methionine, but can be identical to the natural initial amino acid of the product, 2) the protease activity in the periplasm and fermentation medium is distinctly lower than in the cytoplasm, and 3) the formation of any disulfide bridges which are necessary is made possible under the oxidative conditions and by the chaperones in the periplasm.

E. coli has various systems for transporting proteins through the cytoplasmic membrane into the periplasm. The Sec system is used mostly frequently for the secretory production of recombinant proteins. In this case, the gene of the desired protein is functionally linked to a signal sequence of those proteins that are normally exported by E. coli with the aid of the Sec apparatus (e.g. PhoA, OmpA, OmpF, StII, Lpp, MalE). However, it is also possible to use heterologous signal sequences such as, for example, an α-CGTase signal sequence, which are likewise recognized by the Sec apparatus of E. coli (EP0448093). In the case of the Sec system, the proteins are transported into the unfolded state through the cytoplasmic membrane and are folded only subsequently in the periplasm.

The process for producing the recombinant protein is divided into two parts. The first part is the fermentation leading to the crude product. Crude product refers in this context to the result of the fermentation, which comprises the recombinant protein and in addition also contaminating host-specific proteins. The second part of the production process includes the purification of the recombinant protein starting from the crude product.

The complexity and cost of producing recombinant protein are substantially determined by the cost for producing the crude product, which immediately after the fermentation is in the form of a mixture including the recombinant protein and host proteins, and by the cost of purifying the crude product to give the desired recombinant protein. In most cases, the purification takes place over a plurality of stages using chromatographic processes. The depletion of contaminating host proteins, some of which are immunogenic or toxic, is important in the purification process. In this regard, both the intracellular production and the periplasmic production have the following disadvantages:

1. the cells must be disrupted.
2. the target proteins must be purified from a large number of host proteins.

An additional factor regarding intracellular production is that the target proteins are frequently in a form with incorrect folding. Processes that are particularly preferred for producing recombinant proteins in E. coli are therefore those in which the target protein is secreted in high yield and in the correct folding directly into the fermentation medium.

The literature discloses a number of E. coli strains and processes using E. coli strains to achieve secretion of recombinant proteins into the fermentation medium (for review, see Shokri et al., Appl. Microbiol. Biotechnol. 60 (2003), 654-664; Mergulhao et al., Biotechnology Advances 23 (2005), 177-202, Choi and Lee, Appl. Microbiol. Biotechnol. 64 (2004), 625-635). EP 0338410 and EP 0448093 disclose the production and use of a “secretor mutant” of E. coli that exhibits extensive protein secretion into the fermentation medium. The starting strain which can be used for producing suitable E. coli secretor mutants comprises in particular cells having a mina and/or minB mutation (e.g. DS410) or cells which are mutated in a protein or in a plurality of proteins of the outer membrane (e.g. BW7261). These cells were additionally subjected to a mutagenesis procedure, e.g. by treatment with N-methyl-N′-nitro-N-nitrosoguanidine. This is then followed for example by selection for resistance to D-cycloserine, which is a substance acting on the cell wall, or followed by screening for improved protein secretion by analyzing the halo formation in an amylopectin-azure-agar medium utilizing the secretable, starch-degrading enzyme α-cyclodextrin glycosyltransferase (αCGTase) as an indicator protein. It was possible with a secretor mutant generated in this way to produce heterologous proteins such as, for example, an αCGTase and a hirudin derivative with extracellular yields of, respectively, 240 mg/l and 2.63 g/l. A great disadvantage of these secretor mutants is the complicated production procedure by means of mutagenesis and screening. In addition, the production includes a random mutagenesis step that may lead to unwanted mutations in addition to the desired mutation.

EP 0497757 describes the production of E. coli strains that secrete biologically active, i.e. correctly folded, heterologous proteins into the culture medium. These E. coli strains are treated with mutagenic agents. Mutants that have alterations in the outer membrane are sought via resistance to bacteriophage T7 and are tested for the property of “protein secretion into the medium”. The protein yields achieved in the medium with such strains are, however, very low (<5 mg/l). In this case too, there is a further disadvantage in the complicated and poorly reproducible production of such strains.

The approaches described in the prior art exhibit at least one of the following disadvantages:

a) it is usually only possible with a single production system to produce either homologous or very specific proteins extracellularly in sufficiently high yield, or
b) if a system is suitable in principle for producing different types of proteins, only low yields from the economic viewpoint have been achieved therewith to date, or
c) the culturing must be followed by further steps such as, for example, elimination of the target protein from a fusion partner, making the working up more complicated, or
d) the generation of a secretor strain able to secrete proteins with high yield into the fermentation medium is possible only by a complicated mutagenesis and screening process.

In addition, it is possible in principle to use so-called leaky strains. Such strains are mutants of E. coli or Salmonella, which have a defect in the outer membrane thereby releasing periplasmic proteins partly into the fermentation medium. A nonspecific mechanism is involved here (Lazzaroni and Portalier, 1981, J. Bact. 145, 1351-58). Examples of such leaky mutants are strains with altered lipoprotein contents in the outer membrane (e.g. lpp mutants) (Hirota et al., 1977, Proc. Natl. Acad. Sci. USA 74, 1417-20; Yem and Wu, 1978, J. Bact. 133, 1419-26; Suzuki et al., 1978, Mol. Gen. Genet. 167, 1-9).

It is known that lpp mutants release the cell's periplasmic proteins into the fermentation medium (for example, alkaline phosphatase PhoA or RNase I). Such strains are extremely sensitive to EDTA, various detergents and dyes (Fung et al., 1978, J. Bact. 133, 1467-71; Suzuki et al., 1978, Mol. Gen. Genet. 167, 1-9; Hirota et al., 1977, Proc. Natl. Acad. Sci. USA 74, 1417-20).

The use of lpp mutants of E. coli for producing heterologous proteins on an industrial scale has not been described to date. Conversely, it is usually stated that leaky strains of E. coli, which include the lpp mutants, are insufficiently robust and are unsuitable for industrial culturing (EP0357391; Wan and Baneyx, 1998, Protein Expres. Purif. 14, 13-22; Shokri et al., 2003, Appl. Microbiol. Biotechnol. 60: 654-64; Ray et al., 2002, Protein Expres. Purif. 26, 249-59).

Only a few publications that describe the use of lpp mutants on a laboratory scale go beyond pure characterization of these strains. An lpp deletion mutant of E. coli has been employed, because of its property of releasing periplasmic proteins partly into the fermentation medium, as tool for identifying potential virulence genes from pathogenic microorganisms which code for exported proteins having signal peptides which can be eliminated, via screening for halo formation (Giladi et al., 1993, J. Bact. 175, 4129-36). The heterologous target proteins were in this case generated as fusion proteins with E. coli's own periplasmic alkaline phosphatase and secreted as fusion proteins into the fermentation medium.

In another approach, the extracellular accumulation of a fusion protein consisting of the maltose binding protein of E. coli (MBP) and the bacteriocin pediocin AcH (PapA) from Pediococcus acidilactici using an lpp insertion mutant of E. coli has been described (Miller et al., 1998, Appl. Environ. Microbial. 64, 14-20). In this case too, the heterologous target protein was secreted as fusion protein with a protein intrinsic to the cell into the fermentation medium. Both publications describe the production of the fusion proteins in shaken flasks on the laboratory scale in complicated and costly laboratory media (e.g. Luria-Bertani broth).

In addition, Kanamori et al. (1988, Gene 66, 295-300), Morishiva et al. (1994, Thrombosis Research 73, 193-204) and U.S. Pat. No. 5,223,482 disclose the use of the lpp mutant JE5505 (Suzuki et al., 1978, Mol. Gen. Genet. 167, 1-9) for the extracellular production of eukaryotic polypeptides which are composed of a maximum of 70 amino acids and therefore comparatively simple. Moreover, the minimal salt medium M9CA used for the culturing in each case contains, with the supplemented casamino acids, a costly complex component. It was possible in a fermentation process to achieve only low extracellular product yields not exceeding 50 mg/l, which is of no interest for a commercial process, probably attributable to the deficient robustness of the strain under these fermentation conditions.

It has also been described that lpp mutants shed outer membrane vesicles (McBroom and Kuehn, 12.5.2005, section 2.2.4, Outer Membrane Vesicles. In A. Bock, R. Curtiss III, J. B. Kaper, F. C. Neidhardt, T. Nystrom, K. E. Rudd, and C. L. Squires (ed.), EcoSal-Escherichia coli and Salmonella: cellular and molecular biology. [Online.] http://www.ecosal.org. ASM Press, Washington, D.C.) and that the proteins released into the fermentation medium are contained in these vesicles (Kesty and Kuehn, 2004, J. Biol. Chem. 279, 2069-76). The proteins escape the usual process of disulfide bridge formation and the periplasmic isomerization systems necessary for the folding of complex proteins in the periplasm. A typical person skilled in the art would assume that with complex heterologous proteins this leads to incorrect or incomplete folding.

Both production of proteins as fusion proteins, and production of incorrectly folded proteins is unwanted because complicated and costly subsequent treatments of the target protein are therefore necessary. Accordingly, in the case of fusion proteins, the desired target protein must undergo complicated elimination from the fusion partner by specific chemicals or enzymes, and be purified. In the case of incorrectly folded proteins, difficult denaturation and refolding procedures are necessary.

### SUMMARY OF THE INVENTION

It is an object of the present invention to provide a process for producing a heterologous protein on an industrial scale using an E. coli strain in a fermentation medium in which the protein is secreted in high yield into the fermentation medium, and the heterologous protein can be purified without further subsequent treatment directly from the fermentation medium.

This object of the invention is achieved by a process, which comprises fermenting an E. coli strain in a fermentation medium on an industrial scale. The E. coli strain has a mutation in the lpp gene or in the promoter region of the lpp gene, and contains a gene coding for a heterologous protein, which is functionally linked to a signal sequence coding for a signal peptide. The fermentation medium comprises Ca²⁺ ions in a concentration above 4 mg/l or Mg²⁺ ions in a concentration above 48 mg/l, with the E. coli strain secreting the heterologous protein into the fermentation medium, and the protein being removed from the fermentation medium.

### BRIEF DESCRIPTION OF THE DRAWINGS

FIG. 1 shows the vector pKO3-lpp1 from Example 2.

FIG. 2 shows the vector pKO3-lpp3 from Example 3.

FIG. 3 shows the cloning vector pJF118ut from Example 4.

FIG. 4 shows the CGTase expression plasmid pCGT from Example 4.

FIG. 5 shows the interferon α2b expression plasmid PIFN from Example 6.

FIG. 6 shows the plasmid pHC-anti-lysozyme from Example 7.

FIG. 7 shows the Fab expression plasmid pFab-anti-lysozyme from Example 7.

FIG. 8 shows the plasmid pHC-anti-TF from Example 8.

FIG. 9 shows the anti-TF antibody expression plasmid pAK-anti-TF from Example 8.

The abbreviations used in the figures have the following meaning:
tac p/o: tac promoter/operator;
cmR: chloramphenicol resistance;
lpp1: lpp1 allele with base substitution leading to the amino acid exchange Arg77Cys (R77C);
lpp3: lpp3 allele with base substitution leading to the amino acid exchange Gly14Asp (G14D);
M13Ori: M13 origin of replication;
sacB: levan sucrase gene from bacillus;
repA: pSC101 origin of replication, temperature-sensitive;
rrnB: terminator;
bla: β-lactamase gene (ampicillin resistance);
ColE1: ColE1 origin of replication;
TcR: tetracycline resistance gene;
lacIq: repressor of the tac promoter;
cgt-SP: CGTase signal peptide;
CGTase: CGTase gene;
SD: Shine-Dalgarno sequence;
IFNalpha2b: interferonα2b gene;
ompA-SP: ompA signal peptide;
(VH)-CH1: reading frame for the fragment of the heavy chain with the VH and CH1 domains and C-terminal His tag;
(VL)-CL: reading frame for the fragment of the light chain with the VL and CL domains;
His-Tag: His tag at the C terminus of the heavy chain of the Fab fragment;
HC (Anti-TF): reading frame of the heavy chain of the anti-TF antibody;
LC (Anti-TF): reading frame of the light chain of the anti-TF antibody.

### DETAILED DESCRIPTION OF THE PREFERRED EMBODIMENT(S)

The text file Sequence_{—}906_ST25.txt, created Sep. 20, 2007, and of size 15 kilobytes, filed herewith, is hereby incorporated by reference.

In an embodiment of the present invention, a process for forming heterologous proteins is provided. This process comprises fermenting an E. coli strain scale in a fermentation medium on an industrial scale. The E. coli strain has a mutation in the lpp gene or in the promoter region of the lpp gene, and contains a gene coding for a heterologous protein, which is functionally linked to a signal sequence coding for a signal peptide. The fermentation medium comprises Ca²⁺ ions in a concentration above 4 mg/l or Mg²⁺ ions in a concentration above 48 mg/l, with the E. coli strain secreting the heterologous protein into the fermentation medium. The protein formed is removed from the fermentation medium.

High yields means in the context of the present embodiment, protein concentrations in the fermentation medium above 500 mg/l at the end of the culturing or, in the case of proteins which can already be produced with good yield, yields of more than 110% of that which can be produced according to the current state of the art.

E. coli strains having a mutation in the lpp gene are described in the literature (Hirota et al., 1977, Proc. Natl. Acad. Sci. USA 74, 1417-20; Yem and Wu, 1978, J. Bact. 133, 1419-26). In addition, those skilled in the art are aware of methods for generating lpp mutants from any E. coli strains. Such DNA sequences, which differ in their base sequence from the sequence of the wild-type lpp gene owing to mutations are also referred to as lpp alleles. Designations used in the literature as synonyms for the lpp gene are mlpA or lpo.

According, lpp alleles can be transferred, e.g. by transduction using P1 phages or conjugation, from a strain with an lpp mutation to a wild-type lpp strain, the wild-type lpp gene being replaced by the lpp allele.

In addition, those skilled in the art are aware of further methods for generating lpp-alleles. Such lpp alleles are usually, for reasons of simplicity, first generated in vitro and then introduced into the chromosome of the cell, thereby replacing the originally present wild-type lpp gene and therefore generating an lpp mutant. Alleles of the lpp gene can be produced, for example, by nonspecific or targeted mutagenesis with the DNA of the wild-type lpp gene as starting material. Nonspecific mutations within the lpp gene or the promoter region of the lpp gene can be generated by chemical agents such as nitrosoguanidine, ethyl methanesulfonic acid and the like and/or by physical methods and/or by PCR reactions carried out under particular conditions. Methods for introducing mutations at specific positions within a DNA fragment are known. Therefore, one or more bases in a DNA fragment which includes the lpp gene and its promoter region can be replaced by means of PCR using suitable oligonucleotides as primers.

The lpp alleles generated in vitro by the described methods can be introduced into the chromosome of a host cell, instead of the wild-type lpp gene/promoter, by means of simple standard methods. This can take place for example by means of the process described in Link et al. (1997, J. Bacteriol. 179:6228-37) for introducing chromosomal mutations into a gene by the mechanism of homologous recombination. The introduction of a chromosomal deletion of the entire lpp gene or of a part thereof is possible for example with the aid of the λ Red recombinase system by the method described by Datsenko and Wanner (2000, Proc. Natl. Acad. Sci. USA. 97: 6640-5).

The DNA sequence of the lpp gene of E. coli (SEQ ID NO: 1) codes for an Lpp protein having the sequence SEQ ID NO: 2. The first 60 nucleotides therein code for the signal peptide which controls the secretion of the Lpp protein into the periplasm and which is eliminated again during this translocation process. The promoter region of the lpp gene is defined in Inouye and Inouye (1985, Nucleic Acids Res. 13, 3101-10).

The mutation in the lpp gene is preferably a substitution, a deletion or an insertion of one or more nucleotides in the lpp gene or in the promoter region of the lpp gene, leading to the lpp gene no longer being expressed or being expressed to only a reduced extent, or leading to an altered amino acid sequence of the Lpp protein which is associated with a reduction in the functionality of the Lpp protein.

Expression of the lpp gene is reduced owing to a mutation in the sense of the invention when only a maximum of 80% of the amount of Lpp protein is detectable in the cells by comparison with cells of the wild-type strain W3110 (ATTC: 27325). This is possible for example by an immunological quantification of the Lpp protein with the aid of anti-Pal antibodies (Cascales et al., 2002, J. Bacteriol. 184, 754-9).

In addition, those skilled in the art are aware of various methods for determining a reduction in the functionality of the Lpp protein. For example, the appearance of periplasmic proteins of E. coli in the fermentation medium (leakiness), for example measurable by determining the activity of the indicator protein “alkaline phosphatase” released into the fermentation medium, an increased sensitivity to detergents, EDTA or particular dyes, an increased resistance to the antibiotic globomycin, or observation of the formation of so-called blebs in the electron micrograph serve as evidence of a reduced functionality of the Lpp protein (Hirota et al., 1977, Proc. Natl. Acad. Sci. USA 74, 1417-20; Yem and Wu, 1978, J. Bacteriol. 133, 1419-26; Zwiebel et al., 1981, J. Bacteriol. 145, 654-656).

The Lpp functionality in a cell is reduced in the sense of the invention preferably when at least 10% of the total activity of the periplasmic protein “alkaline phosphatase” which is intrinsic to the cell is released, owing to a mutation in the lpp gene or in the promoter region of the lpp gene, during fermentation from the cell into the fermentation medium, or when the resistance of the cells to globomycin is increased by a factor of at least 2 compared with the lpp wild-type strain W3110.

Particularly preferred mutations in the lpp gene are those leading to replacement of the arginine residue at position 77 of SEQ ID NO: 2 by a cysteine residue (lpp1 mutants) and those leading to replacement of the glycine residue at position 14 of SEQ ID NO: 2 by an aspartic acid residue (lpp3 mutants). Additional preferred mutations are those, which, owing to a deletion of at least one nucleotide in the lpp gene itself or in the promoter region of the lpp gene, lead to the cells exhibiting an increased leakiness for periplasmic proteins. Increased leakiness means in this connection that the cells show after fermentation a higher concentration of periplasmic proteins, e.g. of alkaline phosphatase, in the nutrient medium than the E. coli W3110 strain (ATCC 27325).

The proteins, which can be produced in lpp mutants, are heterologous proteins. Heterologous proteins mean proteins, which do not belong to the proteome, i.e. the entire natural protein complement, of an E. coli K12 strain. All proteins naturally occurring in E. coli K12 strains can be derived from the known E. coli K12 genome sequence (Genbank Accession No. NC_{—}000913). The term “heterologous protein” in the sense of the present invention moreover does not include any fusion proteins with an E. coli protein.

The heterologous proteins in this case show more than 50%, preferably more than 70%, more preferably more than 90% of the specific activity or of their effect (function), which is characteristic of the respective heterologous protein.

Preference is given to heterologous, more preferably eukaryotic proteins, which comprise one or more disulfide bridges, or heterologous, and most preferably eukaryotic proteins, which are in the form of dimers or multimers in their functional form. Examples of eukaryotic proteins are antibodies and fragments thereof, cytokines, growth factors, protein kinases and protein hormones.

It is also possible by means of the process of the invention to obtain heterologous proteins which are in the form of dimers or multimers in their functional form, i.e. have a quaternary structure and are composed of a plurality of identical (homologous) or nonidentical (heterologous) subunits, in high yields in the correct active dimeric or multimeric structure from the fermentation medium when its monomeric protein chains are linked to signal peptides for the secretion and are transported by means of the Sec system into the periplasm. This has been possible both with homodimers or multimers, and in the case of heterodimers or -multimers, i.e. with proteins in which the protein chains of the subunits differ in their amino acid sequence. Preferred proteins are those that are composed of a plurality of different protein chains, i.e. represent heterodimers or heteromultimers. This was completely unexpected because with such proteins it is first necessary for the individual protein chains to be transported by means of the Sec system independently of one another into the periplasm in order normally to be folded or assembled there with incorporation of periplasmic enzymes and chaperones into the correct secondary, tertiary and quaternary structure. Heretofore, those skilled in the art have assumed that release of the proteins into the fermentation medium interferes with such complicated folding and assembling processes, and secretion of such proteins in functional form is therefore particularly difficult.

A particularly important class of proteins consisting of a plurality of protein subunits are antibodies. Antibodies are employed in research, in diagnosis and as therapeutic agent on a large scale, so that there is a need for production processes, which are particularly efficient and possible on the industrial scale.

In the case of antibodies, a distinction is made between full-length antibodies and antibody fragments. Full-length antibodies consist of four protein chains, two identical heavy chains and two identical light chains. The various chains are linked together by disulfide bridges. Each heavy chain is composed of a variable region (V_{H}) and of a constant region, which includes the three domains CH1, CH2 and CH3. The region of the heavy chain which includes the CH2 and CH3 domains and which is also referred to as Fc region is not involved in antigen binding, but has other functions such as, for example, activation of the complement system. Each light chain is composed of a variable region (V_{L}) and of a constant region, which includes the C_{L} domain.

Antibodies (immunoglobulins) are assigned to five classes depending on the amino acid sequence of the heavy chain: IgA, IgD, IgE, IgG and IgM. The term full-length antibody means all antibodies in which the light chains in each case include the V_{L} and C_{L} domains, and the heavy chains are substantially composed of the V_{H}-CH1-CH2-CH3 domains. THerefore, the antibody is able to carry out other functions (e.g. activation of the complement system), besides being able to bind a specific antigen,

By contrast, antibody fragments consist only of part of a full-length antibody, normally the part including the antigen-binding site. Examples of antibody fragments are inter alia i) Fab fragments in which the light chains in each case include the V_{L} and C_{L} domains and the heavy chains in each case include the V_{H} and CH1 domains, ii) Fab′ fragments which in principle represent Fab fragments but also have one or more cysteine residues at the C terminus of the CH1 domain, or iii) F(ab′)₂ fragments in which two Fab′ fragments are linked together by disulfide bridges by means of the cysteine residues at the C terminus of the CH1 domain.

E. coli has already been used to produce antibody fragments, but in this case production took place either in the cytoplasm or in the periplasm. It is necessary in both cases for the E. coli cells to be disrupted and for the antibody fragments to be separated from the remaining E. coli proteins.

U.S. Pat. No. 6,204,023 and EP 0396612 describe the extracellular production of Fab fragments. The yields are in the region of a few milligrams per liter. Better et al. (1993, Proc. Natl. Acad. Sci. USA 90, 457-61) describes the extracellular production of chimeric Fab′ and F(ab′)₂ antibody fragments using the E. coli strain W3110ara⁻. The yields of 200-700 mg/l achieved in this case are also too low for a commercial process on the industrial scale.

Heretofore, it has been possible to produce full-length antibodies in the correct quaternary structure in E. coli exclusively in the periplasm (WO02/061090). To obtain the antibodies in this case it is necessary to disrupt the cells. The yields were very low, not exceeding 156 mg/l. Higher yields of up to 880 mg were achieved only when periplasmic folding assistants such as the dsb proteins or FkpA were coexpressed on plasmids in addition to the antibody chains. It was necessary to purify the antibody from the large number of other E. coli proteins.

Experiments within the framework of the present invention surprisingly revealed that extracellular yields of more than 1 g/l are achieved on use of E. coli lpp mutants to produce antibody fragments on the industrial scale. Preferred antibody fragments in this connection are Fab, Fab′ and F(ab′)₂ fragments, particularly preferably Fab fragments.

It was further surprising that extracellular, correctly folded and functional antibodies are obtained in high yields on production of full-length antibodies by means of the process of the invention. Preferred full-length antibodies in this connection are antibodies of the IgG and IgM class, especially of the IgG class.

For secretion of proteins from the cytoplasm into the periplasm it is necessary for the 5′ end of the gene of the protein to be produced to be linked in frame to the 3′ end of a signal sequence for protein export. Suitable for this purpose are in principle the genes of all signal sequences, which make translocation of the target protein possible with the aid of the Sec apparatus in E. coli. Various signal sequences have been described in the prior art, e.g. the signal sequences of the following genes: phoA, ompA, pelB, ompF, ompT, lamB, malE, staphylococcal protein A, StII and others (Choi & Lee, 2004).

Preference is given to the signal sequences of the phoA and ompA gene of E. coli, and particular preference is given to the signal sequence for a cyclodextrin glycosyltransferase (CGTase) from Klebsiella pneumoniae M5al having the sequence SEQ ID NO: 3 (EP0448093).

The DNA molecule, which includes at least one fusion of a signal sequence and the gene of the recombinant target protein is produced by methods known to those skilled in the art. The gene of the target protein can initially be amplified by PCR using oligonucleotides as primers, and subsequently linked by conventional techniques of molecular biology to the DNA molecule which includes the sequence of a signal peptide and which has been generated in an analogous manner to the gene of the target protein, in such a way that an in frame fusion, (i.e. a continuous reading frame including the signal sequence and the gene of the target protein) results. Alternatively, it is also possible to produce the complete DNA molecule, which includes both the abovementioned functional segments by means of gene synthesis. This signal sequence-recombinant gene fusion can then either be introduced into a vector, e.g. a plasmid, or be integrated directly by known methods into the chromosome of the host cell. The signal sequence-recombinant gene fusion is preferably introduced into plasmids.

For secretion of a protein which consists of a plurality of different subunits from the cytoplasm in the periplasm it is necessary for the 5′ end of the respective gene of the subunit to be produced (target gene) and linked to the 3′ end of a signal sequence for protein export. It is possible in this case for the genes of the different subunits to be linked to different or the same signal sequences. Linkage to different signal sequences is preferred, and linkage of one subunit to the signal sequence of the phoA or ompA gene of E. coli, and linkage of the second subunit to the signal sequence for a cyclodextrin glycosyltransferase (CGTase) from Klebsiella pneumoniae having the sequence SEQ ID NO: 3 (EP0448093) is particularly preferred.

The signal sequence-target gene fusions of the individual subunits can then be either introduced into a vector, e.g. a plasmid, or be integrated directly by known methods into the chromosome of the host cell. It is moreover possible for the signal sequence-target gene fusions of the individual subunits to be cloned on separate but mutually compatible plasmids, or they can be cloned on one plasmid. The gene fusions can moreover be combined in one operon or they can be expressed in separate cistrons in each case. Combination in one operon is preferred. It is possible in the same way for the two gene constructs to be integrated into the chromosome of the host cell combined in one operon or in separate cistrons in each case. Again, combination in one operon is preferred.

The DNA expression construct composed of a signal sequence and of a recombinant gene encoding the protein to be secreted is preferably provided with expression signals, which are functional in E. coli (promoter, transcription start, translation start, ribosome binding site, terminator). Suitable promoters are those promoters known to persons skilled in the art. Examples include inducible promoters such as the lac, tac, trc, lambda PL, ara or tet promoter or sequences derived therefrom. Moreover, permanent expression is also possible through the use of a constitutive promoter such as, for example, the GAPDH promoter. However, it is also possible to use a promoter, which is normally linked to the gene of the recombinant protein to be produced.

This expression construct (promoter-signal sequence-recombinant gene) for the protein to be produced is then introduced, using methods known to those skilled in the art, into the cells with an lpp mutation. This takes place for example on a vector, e.g. a plasmid such as, for instance, a derivative of known expression vectors such as pJF118EH, pKK223-3, pUC18, pBR322, pACYC184, pASK-IBA3 or pET. Suitable selection markers for plasmids are genes, which code for a resistance to, for example, ampicillin, tetracycline, chloramphenicol, kanamycin or other antibiotics.

A preferred E. coli strain employed according to the invention is therefore one in which the recombinant gene functionally linked to a signal sequence coding for a signal peptide which is active in E. coli is further provided with expression signals functional in E. coli, preferably a promoter, a transcription start, translation start, a ribosome binding site, and a terminator. The expression signals in this connection are preferably those previously mentioned above.

The culturing (fermentation) of the cells transformed with an expression plasmid takes place on the industrial scale by conventional fermentation processes known to those skilled in the art in a bioreactor (fermenter).

Fermentation preferably takes place in a conventional bioreactor, for example a stirred tank, a bubble column fermenter or an airlift fermenter. A stirred tank fermenter is most preferred. Industrial scale means in the present context a fermenter size, which is sufficient for the production of pharmaceutical proteins in an amount sufficient for clinical tests and for use on patients after authorization of the medicament comprising the pharmaceutical protein. Preference is therefore given to fermenters with a volume of more than 5 l, particularly preferably fermenters with a volume of >50 l.

In the fermentation, the cells of the protein producing strain are cultured in a liquid medium over a period of 16-150 hours, with continuous monitoring and accurate control of various parameters such as, for example, the nutrient supply, the oxygen partial pressure, the pH and the temperature of the culture. The culturing period is preferably 24-72 hours.

Suitable fermentation media are in principle the conventional media known to those skilled in the art for culturing microorganisms.

It is possible in this connection to use complex media or minimal salt media to which a certain proportion of complex components such as, for example, peptone, tryptone, yeast extract, molasses or corn steep liquor is added. Preferred media in this connection are those comprising Ca²⁺, ions in a concentration of more than 4 mg/l up to a maximum of 5000 mg/l, more preferably 10 mg/l to 5000 mg/l, most preferably 40 mg/l-5000 mg/l, or comprising Mg²⁺ ions in a concentration of more than 48 mg/l up to a maximum of 5000 mg/l. Particularly preferred media comprise the Ca²⁺ and Mg²⁺ ions in the stated concentrations.

Preference is given for the production of pharmaceutical proteins (pharmaceutically active proteins) to chemically defined salt media, i.e. media that, in contrast to complete medium, have an accurately defined substrate composition. It is known that the growth of sensitive microorganisms in such media is zero or slow. It was therefore unexpected and surprising that an E. coli strain comprising an lpp mutation and a gene encoding a heterologous protein which is connected in frame to a signal sequence coding for a signal peptide functional in E. coli grows in a defined salt medium comprising Ca²⁺ and Mg²⁺ ions, and secretes large amounts of the heterologous protein into the salt medium.

In this process, the E. coli strain comprising an lpp mutation and a gene encoding a heterologous protein which is connected in frame to a signal sequence coding for a signal peptide functional in E. coli grows, in a fermentation time which is comparably short in relation to a strain without lpp mutation, to comparably high cell densities and moreover secretes large amounts of the heterologous protein into the salt medium. Particularly preferred salt media in this connection are those comprising Ca²⁺ ions in a concentration of more than 4 mg/l up to a maximum of 5000 mg/l, particularly preferably 10 mg/l to 5000 mg/l, very particularly preferably 40-5000 mg/l, or comprising Mg²⁺ ions in a concentration of more than 48 mg/l up to a maximum of 5000 mg/l. Particularly preferred salt media comprise Ca²⁺ and Mg²⁺ ions in the stated concentrations.

In principle, it is possible to use as primary carbon source for the fermentation all sugars, sugar alcohols or organic acids or salts thereof which can be utilized by the cells. Preference is given to the use of glucose, lactose or glycerol. Glucose and lactose are particularly preferred. Combined feeding of a plurality of different carbon sources is also possible. The carbon source can moreover be introduced completely into the fermentation medium at the start of the fermentation, or none or only a part of the carbon source is introduced at the start, and the carbon source is fed in over the course of the fermentation. A particularly preferred embodiment in this connection is one where part of the carbon source is introduced at the start, and part is fed in. It is more preferred for the carbon source to be introduced at the start in a concentration of 10-30 g/l, and for the feeding to be started when the concentration has fallen to less than 5 g/l, and to be designed so that the concentration is kept below 5 g/l.

The oxygen partial pressure (PO₂) in the culture is preferably between 10 and 70% saturation. A PO₂ of between 30 and 60% is preferred, and the PO₂ is more preferably between 45 and 55% saturation.

The pH of the culture is preferably between pH 6 and pH 8. A pH of between 6.5 and 7.5 is preferably adjusted, and the pH of the culture is particularly preferably kept between 6.8 and 7.2.

The temperature of the culture is preferably between 15 and 45° C. A temperature range between 20 and 40° C. is preferred, a temperature range between 25 and 35° C. is more preferred, and 30° C. is most preferred.

Under the stated conditions, E. coli strains comprising an lpp mutation and a gene coding for a heterologous protein which is linked in frame to a signal sequence coding for a signal peptide functional in E. coli grow in a short fermentation time on the production scale, i.e. in a fermenter with a working volume of >5 l, to normal cell densities. Moreover, these strains secrete large amounts of heterologous proteins into the fermentation medium.

The secreted protein can be purified from the crude product by conventional purification methods known to the skilled artisan, as known in the state of the art. In a first step there is normally removal, by separation methods such as centrifugation or filtration, of the cells from the secreted target protein. The target protein can then be concentrated for example by ultrafiltration and is then further purified by standard methods such as precipitation, chromatography or ultrafiltration. Particularly preferred methods in this connection are those such as affinity chromatography in which the already correctly folded native conformation of the protein is utilized.

The following examples serve to explain the invention further. All the methods of molecular biology employed, such as polymerase chain reaction (PCR), gene synthesis, isolation and purification of DNA, modification of DNA by restriction enzymes, Klenow fragment and ligase, transformation etc., were carried out in the manner known to the skilled artisan, described in the literature or recommended by the respective manufacturers.

### EXAMPLE 1

Generation of a Chromosomal lpp Deletion Mutant From a Wild-type E. coli Strain

The procedure for generating an lpp deletion mutant of the wild-type E. coli strain W3110 (American Type Culture Collection (ATCC): 27325) with the aid of λ recombinase was according to the method of Datsenko and Wanner (2000, Proc. Natl. Acad. Sci. USA. 97: 6640-5). This entailed initially generating, with the aid of the polymerase chain reaction (PCR) using the oligonucleotides lpp1 (SEQ ID NO: 4) and lpp2 (SEQ ID NO: 5) as primers and the plasmid pKD3 (Coli Genetic Stock Center (CGSC): 7631) as template, a linear DNA fragment which comprises a chloramphenicol resistance gene and which is flanked by in each case 50 base pairs of the upstream region and of the downstream region of the lpp gene.

The strain W3110 was firstly transformed with the plasmid pKD46 (CGSC: 7739). Competent cells of the strain W3110 pKD46 obtained in this way, which had been produced in accordance with the statements of Datsenko and Wanner, were transformed with the linear DNA fragment generated by PCR. Selection for integration of the chloramphenicol resistance cassette into the chromosome of W3110 at the position of the lpp gene took place on LB agar plates containing 20 mg/l chloramphenicol. Cells in which the lpp gene had been virtually completely replaced by the chloramphenicol resistance cassette were obtained in this way. PCR using the oligonucleotides pykF (SEQ ID NO: 6) and ynhG2 (SEQ ID NO: 7) and chromosomal DNA of the chloramphenicol-resistant cells as template confirmed integration at the correct position in the chromosome.

The cells were cured of the plasmid pKD46 by the described procedure (Datsenko and Wanner), and the strain generated in this way was called W3110lpp::cat. Deletion of the chloramphenicol resistance cassette from the chromosome of the strain W3110lpp::cat took place according to the protocol of Datsenko and Wanner with the aid of the plasmid pCP20 (CGSC: 7629), which harbors the FLP recombinase gene. The chloramphenicol-sensitive lpp deletion mutant of W3110 finally obtained by this procedure was called W3110Δlpp.

### EXAMPLE 2

Generation of a Chromosomal lpp1 Mutant From a Wild-type E. coli Strain

Replacement of the wild-type lpp gene in the chromosome of the strain W3110 by the lpp1 allele took place by homologous recombination. The procedure for this was as follows:

A DNA molecule which contains the lpp1 allele and about 200 base pairs of the DNA region located on the 3′ side of the wild-type lpp gene (SEQ ID NO: 8) were produced by gene synthesis. This DNA molecule also has at each of the two ends a cleavage site for the restriction enzyme BamHI. The lpp1 allele includes bases 9 to 245 of SEQ ID NO: 8. The lpp1 allele differs from the wild-type lpp gene (SEQ ID NO: 1) by having a base substitution at position 229 (C to T) of the lpp gene, leading to replacement of the arginine residue at position 77 by a cysteine residue in the unprocessed Lpp protein.

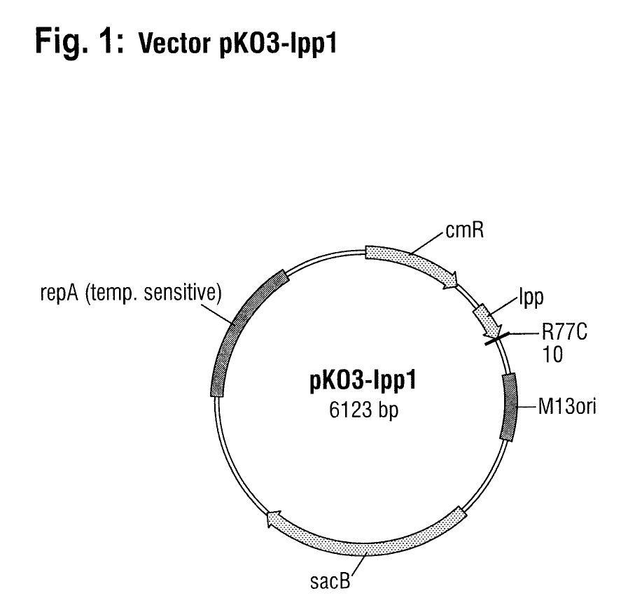

The DNA molecule generated by gene synthesis and having SEQ ID NO: 8 was cut completely with the restriction enzyme BamHI. The cloning vector pKO3 (Link et al., 1997, J. Bacteriol. 179, 6228-37; Harvard Medical School, Department of Genetics, 200 Longwood Ave, Boston, MA 02115) was initially likewise cut with the restriction enzyme BamHI. The plasmid linearized in this way was then treated with alkaline phosphatase in order to prevent later re-ligation of the vector. The two DNA molecules cut in this way were ligated together. The plasmid generated in this way was called pKO3-lpp1 (FIG. 1).

The strain W3110 was transformed by the CaCl₂ method with the plasmid pKO3-lpp1, with plasmid-harboring cells being selected using ampicillin. Subsequent replacement of the wild-type lpp gene by the lpp1 allele took place by the homologous recombination mechanism using the procedure described in Link et al. (1997). It was checked that this exchange took place precisely at the correct base position in the chromosome by first amplifying the chromosomal lpp region by PCR using the oligonucleotides pykF (SEQ ID NO: 6) and ynhG2 (SEQ ID NO: 7) and chromosomal DNA of the putative lpp1 mutant as template, and then sequencing the PCR product using the same oligonucleotides. The lpp1 mutant of W3110 finally generated in this way was called W3110lpp1.

### EXAMPLE 3

Generation of a Chromosomal lpp3 Mutant From a Wild-type E. coli Strain

The procedure for generating a chromosomal lpp3 mutant of W3110 which, like the lpp1 mutant, has only one point mutation in the lpp gene was analogous to Example 2, with the difference that a DNA molecule with SEQ ID NO: 9, which was likewise produced by gene synthesis, was used instead of the DNA fragment with SEQ ID NO: 8. This DNA molecule comprises the lpp3 allele (bases 211 to 447) and about 200 base pairs of the DNA region located on the 5′ side of the wild-type lpp gene. This DNA molecule additionally has at each of the two ends a cleavage site for the restriction enzyme BamHI. The lpp3 allele differs from SEQ ID NO: 1 by having a base substitution at position 41 (G to A) of the lpp gene, leading to replacement of the glycine residue at position 14 by an aspartic acid residue in the as yet unprocessed Lpp protein.

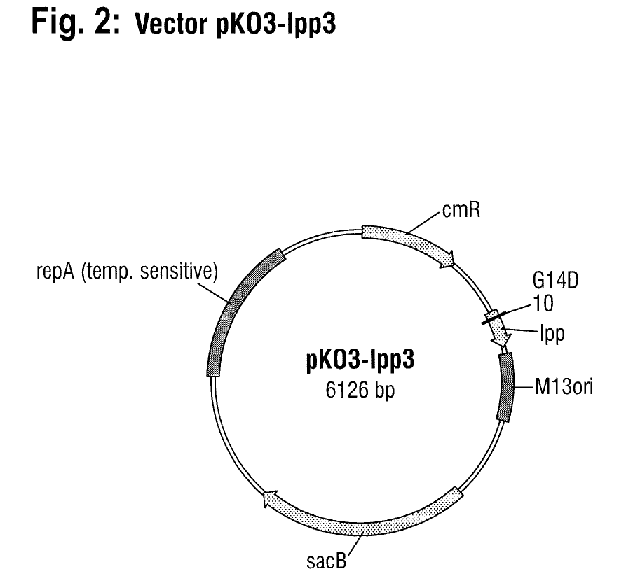

The plasmid pKO3-lpp3 (FIG. 2) generated by ligation of the respectively BamHI-cut DNA fragments of plasmid pKO3 and the DNA molecule containing the lpp3 allele was transformed into the strain W3110 as described above. Finally, the strain W3110lpp3 was obtained by the procedure of Link et al. The strain was checked as described in Example 2.

### EXAMPLE 4

Fermentative Production of a Cyclodextrin Glycosyltransferase with lpp Mutants on the 10 1 Scale

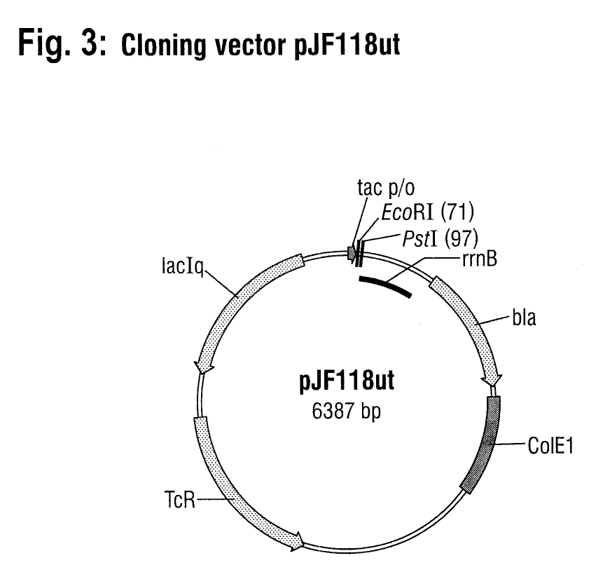

A DNA fragment with SEQ ID NO: 10, which comprises a cyclodextrin glycosyltransferase (CGTase) gene from Klebsiella pneumoniae M5al (Genbank No. M15264) was produced by gene synthesis. This DNA fragment was cloned into the expression vector pJF118ut (FIG. 3) which is deposited at the DSMZ-Deutsche Sammlung von Mikroorganismen und Zellkulturen GmbH (Braunschweig) under the number DSM 18596. pJF118ut is a derivative of the well-known expression vector pKK223-3 (Amersham Pharmacia Biotech) and comprises besides the β-lactamase gene and the tetracycline resistance gene also the tac promoter, which is repressed by the LacIq gene product, whose gene is likewise present on the plasmid, and which can be switched on by an inducer such as, for example, D-lactose or isopropyl β-D-thiogalactopyranoside (IPTG).

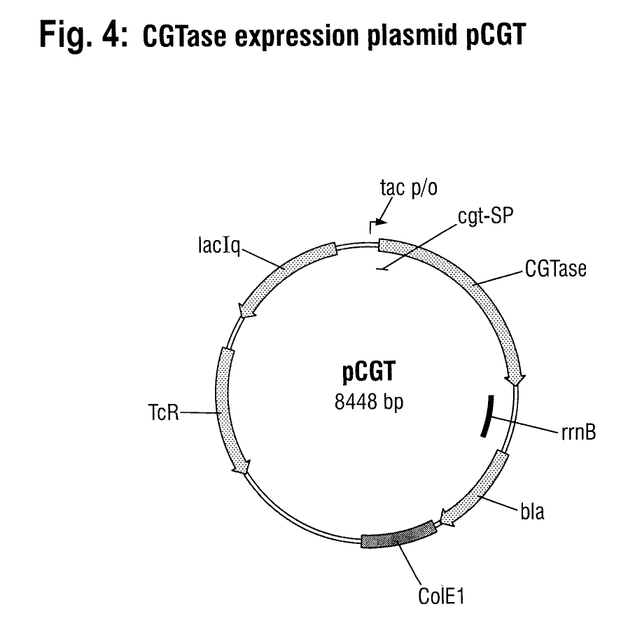

The plasmid pJF118ut was completely cut with the restriction enzyme EcoRI, and the bases protruding in each case at the 5′ ends of the linear DNA fragment were eliminated with S1 nuclease. The vector DNA molecule prepared in this way was ligated to the CGTase-including DNA fragment (SEQ ID NO: 10) using T4 ligase. The strain DH5α was transformed with the ligation mixture by the CaCl₂ method, selecting for plasmid-containing cells using ampicillin (100 mg/l). The plasmid was reisolated from ampicillin-resistant transformants and checked by restriction analysis. The plasmid generated in this way, in which expression of the CGTase gene is under the control of the tac promoter, was called pCGT (FIG. 4).

To produce a cyclodextrin glycosyltransferase on the 10 l scale, the strains W3110Δlpp, W3110lpp1 and W3110lpp3 were in each case transformed with the PCGT plasmid by the CaCl₂ method. Ampicillin (100 mg/l) was used to select for plasmid-containing cells.

Production was carried out in 10 l stirred tank fermenters. The fermenter charged with 6 l of the fermentation medium FM4 (1.5 g/l KH₂PO₄; 5 g/l (NH₄)₂SO₄; 0.5 g/l MgSO₄×7 H₂O; 0.15 g/l CaCl₂×2 H₂O, 0.075 g/l FeSO₄×7 H₂O; 1 g/l Na₃ citrate×2 H₂O; 0.5 g/l NaCl; 1 ml/l trace element solution (0.15 g/l Na₂MoO₄×2 H₂O; 2.5 g/l Na₃BO₃; 0.7 g/l CoCl₂×6 H₂O; 0.25 g/l CuSO₄×5 H₂O; 1.6 g/l MnCl₂×4 H₂O; 0.3 g/l ZnSO₄×7 H₂O); 5 mg/l vitamin B₁; 3 g/l Phytone; 1.5 g/l yeast extract; 10 g/l glucose; 100 mg/l ampicillin) was inoculated in the ratio 1:10 with a preculture which was cultured in the same medium overnight. A temperature of 30° C. was set during the fermentation, and the pH was kept constant at a value of 7.0 by metering in NH₄OH or H₃PO₄. Glucose was metered in throughout the fermentation, aiming at a maximum glucose concentration of <10 g/l in the fermentation medium. Expression was induced by adding isopropyl β-D-thiogalacto-pyranoside (IPTG) ad 0.1 mM at the end of the logarithmic growth phase.

After fermentation for 72 h, samples were taken, the cells were removed from the fermentation medium by centrifugation, and the CGTase content in the fermentation supernatant was determined by the following activity assay: Assay buffer: 5 mM Tris-HCl buffer>pH 6.5, 5 mM CaSO₄·2H₂O Substrate: 10% strength Noredux solution in assay buffer (pH 6.5)

Assay mixture: 1 ml of substrate solution+1 ml of centrifuged and, where appropriate, diluted culture supernatant (5 min., 12,000 rpm)+3 ml of methanol Reaction temperature: 40° C.

Enzyme assay:
Pre-equilibration of the solutions (about 5 min at 40° C.)
Addition of the enzyme solution to the substrate solution; rapid mixing (whirl mixer)
Incubation at 40° C. for 3 min.
Stopping of the enzyme reaction by addition of methanol; rapid mixing (whirl mixer)
Cooling of the mixture on ice (about 5 min).
Centrifugation (5 min, 12,000 rpm) and removal of the clear supernatant by pipette
HPLC analysis of the CD produced
   Enzymic activity: A=C*D1*D2/(t*MW) (units/ml)
   A=activity
   C=content of CD in mg/l=assay mixture: area units×104/standard solution (10 mg/ml)/area units
   D1=dilution factor in the assay mixture (when carried out as stated above: D1=5)
   D2=dilution factor of the culture supernatant before being used in the assay; if undiluted: D2=1
   t=reaction time in min.
   MW=molecular weight in g/mol (CD=973 g/mol)
   1 unit=1 μmol of product/min.
   The amount of CGTase present in the fermentation supernatant can be calculated from the CGTase activity determined in this way. In this connection, 150 U/ml CGTase activity are equivalent to about 1 g/l CGTase protein.

Table 1 shows the yields of cyclodextrin glycosyltransferase obtained in each case.

TABLE 1
Yields of cyclodextrin glycosyltransferase in the fermentation
supernatant after fermentation for 72 h

| | Cyclodextrin | Cyclodextrin |
|---|---|---|
| | glycosyl- | glycosyl- |
| | transferase | transferase |
| Strain | (U/ml) | (g/l) |
| W3110Δlpp/pCGT | 480 | 3.2 |
| W3110lpp1/pCGT | 450 | 3.0 |
| W3110lpp3/pCGT | 520 | 3.5 |

### EXAMPLE 5

### Fermentative Production of a Hirudin Derivative With lpp Mutants on the 10 1 Scale

Hirudin is a polypeptide having 65 amino acids and was originally isolated from the leech Hirudo medicinalis. This example describes the fermentative production of a hirudin derivative having the N-terminal amino acid sequence Ala-Thr-Tyr-Thr-Asp.

To produce the hirudin derivative, the strains W3110Δlpp, W3110lpp1 and W3110lpp3 were each initially transformed with the plasmid pCMT203 by the CaCl₂ method. Ampicillin (100 mg/l) was used to select for plasmid-containing cells.

The plasmid pCMT203 comprises besides the gene for ampicillin resistance inter alia also the structural gene of the hirudin derivative which is fused in frame to the 3′ end of a CGTase signal sequence. Expression of the CGTase signal sequence-hirudin fusion is under the control of the tac promoter. The plasmid pCMT203 is described in EP0448093B1.

Production of the hirudin derivative on the 10 l scale took place in analogy to the process described in Example 4 using the strains W3110Δlpp/pCMT203, W3110lpp1/pCMT203 and W3110lpp3/pCMT203. After fermentation for 72 h, samples were taken, and then the cells were removed from the fermentation medium by centrifugation, and the hirudin content in the fermentation supernatant was determined by a thrombin inactivation assay as described in Chang (1991, J. Biol. Chem. 266, 10839-43). In this case, 16,000 antithrombin (AT) unit/ml hirudin activity are equivalent to about 1 g/l hirudin protein.

Table 2 shows the hirudin yields in the fermentation supernatant obtained in each case.

TABLE 2
Hirudin yield (in AT-U/ml and g/l) in the fermentation
supernatant after fermentation for 72 h.

| | Hirudin | |
|---|---|---|
| Strain | (AT-U/ml) | Hirudin (g/l) |
| W3110Δlpp/pCMT203 | 50000 | 3.1 |
| W3110lpp1/pCMT203 | 44000 | 2.8 |
| W3110lpp3/pCMT203 | 48000 | 3.0 |

### EXAMPLE 6

Fermentative Production of Interferonα2b with lpp Mutants on the 10 l Scale

A further protein of pharmaceutical interest which can be produced extracellularly with the aid of an lpp mutant of E. coli is interferonα2b.

The procedure for generating the expression vector for the interferonα2b gene was as follows:

A DNA fragment with SEQ ID NO: 11 which comprises a gene fusion consisting of the CGTase signal sequence (SEQ ID NO: 3) described in EP 0220714 and the gene for interferonα2b was produced by gene synthesis.

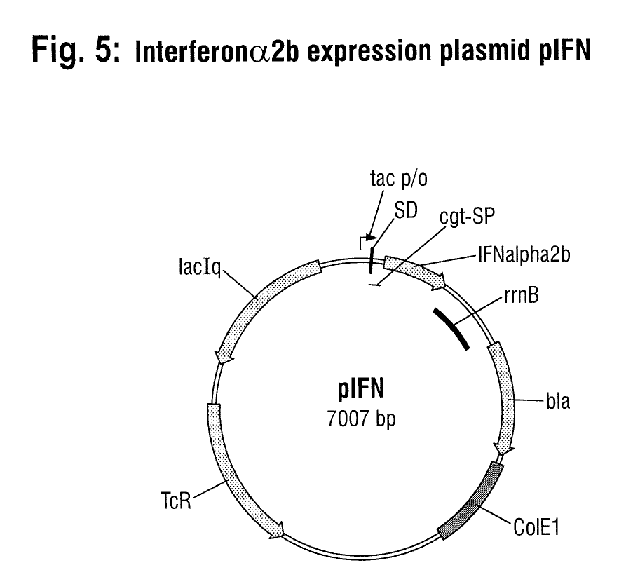

This DNA fragment was cut with the restriction enzymes EcoRI and PstI and ligated to the expression vector pJF118ut which had been cut with the same restriction enzymes. The plasmid resulting from this cloning, in which expression of the interferonα2b gene is under the control of the tac promoter was called PIFN (FIG. 5).

To produce interferonα2b, the strains W3110Δlpp, W3110lpp1 and W3110lpp3 were each transformed with the plasmid PIFN by the CaCl₂ method. Ampicillin (100 mg/l) was used to select for plasmid-containing cells.

Production of interferonα2b on the 10 l scale took place in analogy to the process described in Example 4 with the strains W3110Δlpp/pIFN, W3110lpp1/pIFN and W3110lpp3/pIFN. After fermentation for 72 h, samples were taken and then the cells were removed from the fermentation medium by centrifugation, and the interferonα2b content in the fermentation supernatant was determined.

For this purpose, the proteins in the fermentation supernatant were fractionated by electrophoresis in an SDS polyacrylamide gel and quantified by detection in an immunoblot with anti-interferon-specific antibodies as follows:

1 μl of supernatant was mixed with sample buffer (2×Tris SDS—sample buffer (Invitrogen Cat. No. LC2676): 0.125 M Tris.HCl, pH 6.8, 4% w/v SDS, 20% v/v glycerol, 0.005% v/v bromophenol blue, 5% beta-mercaptoethanol). In addition, defined amounts of interferonα2b were also loaded as standard. The proteins were denatured by heating at 100° C. for 5 min, cooling on ice for 2 min and centrifuging. The proteins were fractionated by electrophoresis in a 12% NuPAGE® Bis-Tris gel (Invitrogen Cat. No. NP0341) with 1×MES-containing running buffer (Invitrogen Cat. No. NP0002) (electrophoresis parameters: 40 min. at 200 V). Detection and quantification by immunoblotting was carried out in accordance with the following protocol:

### Transfer in a Wet Blotting Method:

Module: Amersham: Hoefer TE 22 Mini Tank Transfer Unit, Code Number: 80-6204-26

Membrane: nitrocellulose membrane (Schleicher&Schuell, BA 85, cellulose nitrate (E), 0.45 μm pore size)

Cut Whatman filter and nitrocellulose membrane to the appropriate size and submerge with pieces of foam (sponges) in transfer buffer (Invitrogen Cat. No. LC3675) free of air bubbles.

Assembly of the sandwich: black grid, connection to the cathode, 2 sponges, each 3 mm thick, Whatman paper, SDS polyacrylamide gel, NC membrane, Whatman, 1 sponge, 6 mm thick, white grid, connection to the anode.

Transfer conditions: I=200 mA constant current,
U=unlimited, running time 60 min.

### Prehybridization

Incubation of the membrane in 25 ml of prehybridization buffer Swirl at RT for 30 min

### Hybridization of 1^{st} Antibody

Incubation of the membrane in 25 ml prehybridization buffer+0.15 μg/ml (→3.75 μg) anti-human IFN-alpha antibody (Pepro Tech EC, through Biozol Cat. No.: 500-P32A)
Swirl at RT for 90 min or overnight

### Washing

Swirl with 1×PBS for 10 seconds, RT, pour off buffer
Swirl with 1×PBS for 2×15 min, RT, pour off buffer

### Hybridization of 2^{nd} Antibody

Incubation of the membrane in 25 ml of prehybridization buffer+25 μl (1:1000) of goat anti-rabbit IgG horseradish peroxidase conjugate (HRP) (Southern Biotech, through Biozol Cat. No.: 4050-05)

### Swirl at RT for 60 min

### Washing

Swirl with 1×PBS for 10 seconds, RT, pour off buffer
Swirl with 1×PBS for 2×15 min, RT, pour off buffer

### Detection via Chemiluminescence

Prepare Lumi-Light Western blotting substrate (Roche, Cat. No.: 2015200): mix Lumi-Light luminol/enhancer solution and Lumi-Light stable peroxide solution in the ratio 1:1:3 ml/NC membrane.

Incubate blot with Lumi-Light Western blotting substrate at RT for 5 min, drain off excess, cover membrane with plastic wrap and immediately lay on an X-ray film (Kodak, X-OMAT), expose for 2 min, develop and fix. If the signals are weak, the exposure is repeated over a longer period.

### Buffers

Prehybridization buffer: 5% skimmed milk powder in 1×PBS 10×PBS: 100 mM NaH₂PO₄, 1.5 M NaCl, pH 7.5 with NaOH, 0.5% Triton 100
1×PBS: dilute 10×PBS 1:10 with deionized water

### Quantification

A quantitative evaluation took place after scanning of the immunoblot with a Biorad GS-800 calibrated densitometer using the quantity One l-D-analysis Software (Biorad) by comparison with the loaded standard.

The interferonα2b yields determined in this way in the fermentation supernatant are depicted in Table 3.

TABLE 3
Interferonα2b yields in the fermentation
supernatant after fermentation for 72 h

| Strain | Interferonα2b (mg/l) |
|---|---|
| W3110Δlpp/pIFN | 530 |
| W3110lpp1/pIFN | 510 |
| W3110lpp3/pIFN | 570 |

### EXAMPLE 7

### Fermentative Production of Fab Antibody Fragments With lpp Mutants on the 10 l Scale

Extracellular production of functional Fab antibody fragments is also possible with the aid of an lpp mutant of E. coli. In this case, the cell must simultaneously synthesize the corresponding fragments of the light chain which includes the V_{L} and C_{L} domains, and of the heavy chain which includes the V_{H} and CH1 domains, and then secrete them into the periplasm and finally into the fermentation medium. The two chains are then assembled to give the functional Fab fragment outside the cytoplasm.

The present example describes the production of an Fab fragment of the well-characterized anti-lysozyme antibody D1.3. The plasmid pJF118ut served as starting vector for cloning and expression of the genes of the anti-lysozyme Fab fragment. The two reading frames for the heavy chain (V_{H}-C_{H}1 domains) and for the light chain (V_{L}-C_{L} domains) of the anti-lysozyme Fab fragment, in each case including a signal sequence, were cloned into this plasmid in two consecutive steps. The procedure for this was as follows:

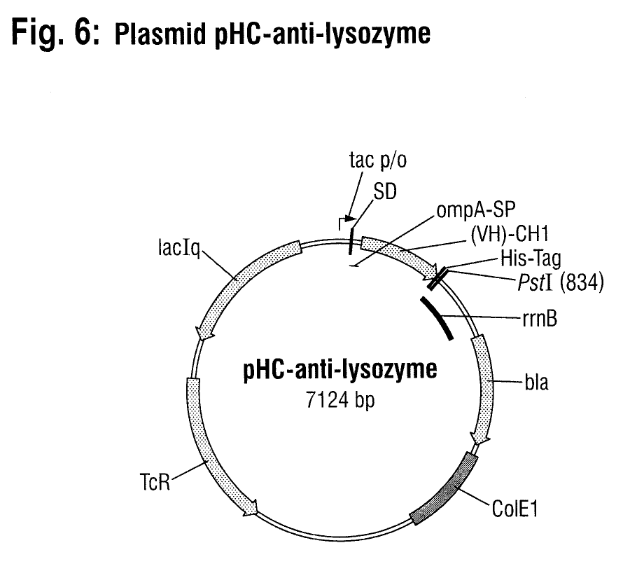

The DNA fragment with SEQ ID NO: 12 (heavy chain) was produced by gene synthesis and includes a gene fusion consisting of the signal sequence of the ompA gene of E. coli and of the reading frame for the heavy chain (V_{L}-CH1) of the Fab fragment. Six histidine codons are directly connected to this reading frame and thereby forming the C terminus of the fusion protein. Simple purification of the completely assembled Fab fragment by affinity chromatography is subsequently possible via this His tag. This DNA fragment was cut with the restriction enzymes EcoRI and PstI and ligated to the expression vector pJF118ut which had been cut with the same restriction enzymes. The plasmid resulting from this cloning, in which expression of the gene for the heavy chain is under the control of the tac promoter, was called pHC-anti-lysozyme (FIG. 6).

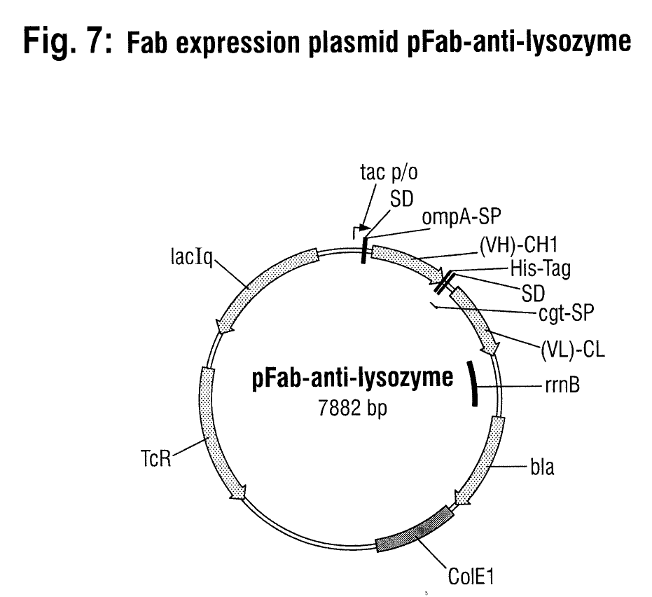

The DNA fragment with SEQ ID NO: 13 (light chain) was likewise produced by gene synthesis and includes a gene fusion consisting of the signal sequence of a CGTase (SEQ ID NO: 3) and of the reading frame for the light chain (V_{L}-C_{L}) of the Fab fragment. This DNA fragment was firstly cut with the restriction enzyme PstI and then ligated to the vector pHC-anti-lysozyme, which had been cut with the same restriction enzyme. The plasmid resulting therefrom was called pFab-anti-lysozyme (FIG. 7). An artificial operon which consists of the respective reading frames for the heavy and the light chain and which is under the control of the tac promoter was generated in this way. Synchronous expression of the two genes is possible by adding an inducer (e.g. IPTG).

To produce the anti-lysozyme Fab fragment, the strains W3110Δlpp, W3110lpp1 and W3110lpp3 were each transformed with the plasmid pFab-anti-lysozyme by the CaCl₂ method. Ampicillin (100 mg/l) was used to select for plasmid-containing cells.

Production of the anti-lysozyme Fab fragment on the 10 l scale took place in analogy to the process described in Example 4 with the strains W3110Δlpp/pFab-anti-lysozyme, W3110lpp1/pFab-anti-lysozyme and W3110lpp3/pFab-anti-lysozyme. After fermentation for 72 h, samples were taken and then the cells were removed from the fermentation medium by centrifugation.

The anti-lysozyme Fab fragment was purified from the fermentation supernatants by affinity chromatography as described in Skerra (1994, Gene 141, 79-84).

Quantification and determination of the activity of the purified anti-lysozyme Fab fragment took place by an ELISA assay with lysozyme as antigen (Skerra, 1994, Gene 141, 79-84).

Table 4 lists the yields of functional anti-lysozyme Fab fragment which could each be isolated from 20 ml portions of fermentation supernatant after fermentation for 72 hours.

TABLE 4
Anti-lysozyme Fab fragment yields in the fermentation
supernatant after fermentation for 72 h

| | | Anti-lysozyme Fab |
|---|---|---|
| | | fragment yield |
| | Anti-lysozyme Fab | [g/l] in the |
| | fragment purified | fermentation |
| | from 20 ml of | supernatant |
| Strain | supernatant [mg] | (extrapolated) |
| W3110Δlpp/ | 27 | 1.3 |
| pFab-Anti-Lysozyme | | |
| W3110lpp1/ | 20 | 1.0 |
| pFab-Anti-Lysozyme | | |
| W3110lpp3/ | 30 | 1.5 |
| pFab-Anti-Lysozyme | | |

### EXAMPLE 8

### Fermentative Production of Full-length Antibodies With lpp Mutants on the 10 l Scale

Extracellular production of functional full-length antibodies is also possible with the aid of an lpp mutant of E. coli. In an analogous manner to the production of the Fab fragments, the cell must synthesize the light and the heavy chain of the antibody simultaneously and then secrete them into the periplasm and finally into the fermentation medium. Assembling of the two chains to form the functional full-length antibody then takes place outside the cytoplasm. The present example describes the production of the anti-tissue factor (αTF) IgGl antibody.

The plasmid pJF118ut served as starting vector for the cloning and expression of the genes of the anti-αTF antibody. The two reading frames for the heavy chain and for the light chain of the anti-αTF antibody, in each case including a signal sequence, were cloned into this plasmid in two consecutive steps.

The procedure for this was as follows:

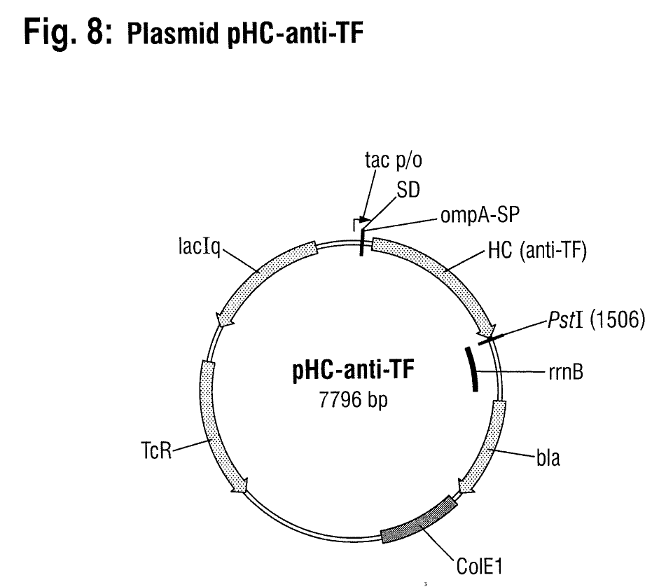

The DNA fragment with SEQ ID NO: 14 (heavy chain) was produced by gene synthesis and includes a gene fusion consisting of the signal sequence of the ompA gene of E. coli and of the reading frame for the heavy chain of the anti-αTF antibody. This DNA fragment was initially cut with the restriction enzymes EcoRI and PstI and ligated to the expression vector pJF118ut which had been cut with the same restriction enzymes. The plasmid resulting from this cloning, in which expression of the gene for the heavy chain is under the control of the tac promoter, was called pHC-anti-TF (FIG. 8).

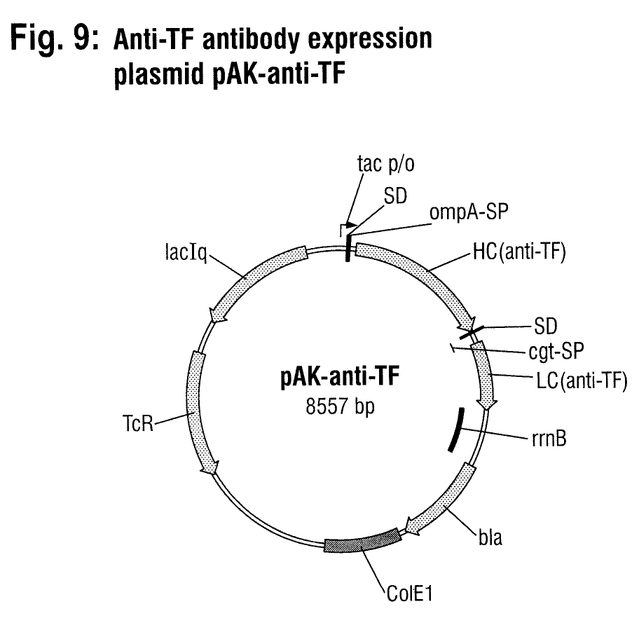

The DNA fragment with SEQ ID NO: 15 (light chain) was likewise produced by gene synthesis and includes a gene fusion consisting of the signal sequence of a CGTase (SEQ ID NO: 3) and of the reading frame for the light chain of the anti-αTF antibody. This DNA fragment was initially cut with the restriction enzyme PstI and then ligated to the vector pHC-anti-TF which had been cut with the same restriction enzyme. The plasmid resulting therefrom was called pAK-Anti-TF (FIG. 9). An artificial operon which consists of the respective reading frames for the heavy and the light chain and which is under the control of the tac promoter was generated in this way. Synchronous expression of the two genes is possible by adding an inducer (e.g. IPTG).

To produce the anti-αTF antibody, the strains W3110Δlpp, W3110lpp1 and W3110lpp3 were each transformed with the plasmid pAK-anti-TF by the CaCl₂ method. Ampicillin (100 mg/l) was used to select for plasmid-containing cells.

Production of the anti-αTF antibody on the 10 l scale took place in analogy to the process described in Example 4 with the strains W3110Δlpp/pAK-anti-TF, W3110lpp1/pAK-anti-TF and W3110lpp3/pAK-anti-TF. After fermentation for 72 h, samples were taken and then the cells were separated from the fermentation medium by centrifugation.

Quantification of the anti-αTF antibody secreted into the fermentation medium took place by determining the activity using an ELISA assay with soluble tissue factor as antigen (coating) and a peroxidase-conjugated goat anti-human F(ab′)₂ fragment as secondary antibody, as described in Simmons et al. (2002, J. Immunol. Methods 263, 133-47).

Table 5 lists the yields of functional anti-αTF antibody determined in this way.

TABLE 5
Anti-αTF antibody yields in the fermentation
supernatant after fermentation for 72 h

| Strain | Anti-αTF antibody [mg/l] |
|---|---|
| W3110Δlpp/pAK-Anti-TF | 580 |
| W3110lpp1/pAK-Anti-TF | 600 |
| W3110lpp3/pAK-Anti-TF | 650 |

What is claimed is:

1. A method for producing a heterologous protein by utilizing E. coli in a fermentation medium, the method comprising:
a) fermenting an E. coli strain in a fermentation medium such that the E. coli strain secretes the heterologous protein into the fermentation medium, the E. coli strain having:
a mutation in the lpp gene or in the promoter region of the lpp gene; and
a gene coding for a heterologous protein that is functionally linked to a signal sequence coding for a signal peptide;
the fermentation medium comprising Ca²⁺ ions in a concentration above 4 mg/l or Mg²⁺ ions in a concentration above 48 mg/l; and
b) removing the protein from the fermentation medium.

2. The method of claim 1, wherein the mutation in the lpp gene is a substitution, a deletion or an insertion of one or more nucleotides in the lpp gene or in the promoter region of the lpp gene, leading to the lpp gene no longer being expressed or being expressed to only a reduced extent, or leading to an altered amino acid sequence of the Lpp protein which is associated with a reduction in the functionality of the Lpp protein.

3. The method of claim 1, wherein the mutation in the lpp gene brings about replacement of the arginine residue at position 77 of SEQ ID NO: 2 by a cysteine residue (lpp1 mutants) leading to cells exhibiting an increased leakiness for periplasmic proteins.

4. The method of claim 1, wherein the mutation in the lpp gene brings about a replacement of the glycine residue at position 14 of SEQ ID NO: 2 by an aspartic acid residue (lpp3 mutants) leading to the cells exhibiting an increased leakiness for periplasmic proteins.

5. The method of claim 1, wherein the mutation in the lpp gene includes a deletion of at least one nucleotide in the lpp gene itself or in the promoter region of the lpp gene leading to the cells exhibiting an increased leakiness for periplasmic proteins.

6. The method of claim 1 wherein the protein comprises one or more disulfide bridges.

7. The method of claim 1, wherein the protein is in its functional form a dimer or multimer.

8. The method of claim 1, wherein the heterologous protein is a eukaryotic protein.

9. The process as claimed in claim 8, wherein the eukaryotic protein is an antibody or antibody fragment, a cytokine, a growth factor, a protein kinase or a protein hormone.

10. The method of claim 9, wherein an antibody fragment is produced in an extracellular yield of more than 1 g/l.

11. The method of claim 1 wherein the gene coding for a signal peptide is selected from the group consisting of genes coding for the signal sequence of the phoA or ompA gene of E. coli and the signal sequence having SEQ ID NO: 3.

12. Method for the secretion of a protein consisting of several subunits according to claim 6, wherein the 5′ end of the gene of the protein subunit to be produced is linked in frame to the 3′ end of a signal sequence for protein export, with the genes of the different subunits of the protein being linked to different signal sequences.

13. The method of claim 1 wherein the fermentation takes place in a fermenter with a volume of more than 5 l.

14. The method of claim 1, wherein the fermentation medium comprises Ca²⁺ ions in a concentration of from above 4 mg/l up to 5000 mg/l.

15. The method of claim 1, wherein the fermentation medium comprises Ca ions in a concentration of from above 10 mg/l up to 5000 mg/l.

16. The method of claim 1, wherein the fermentation medium comprises Ca² ions in a concentration of from above 40 mg/l up to 5000 mg/l.

17. The method of claim 1, wherein the fermentation medium comprises Mg²⁺, ions in a concentration from above 48 mg/l up to 5000 mg/l.

18. The method of claim 1, wherein the fermentation medium is a minimal salt medium.

19. The method of claim 1, wherein the fermentation takes place over a period of 24 hours to 72 hours.


SEQUENCE LISTING (15 sequences)

SEQ ID NO: 1 - DNA, 237 bp, Escherichia coli
  gene (1)..(237): Wild-type lpp gene
atgaaagctactaaactggtactgggcgcggtaatcctgggttctactctgctggcaggttgctccagcaacgctaaaatcgatcagctgtcttctgacgttcagactctgaacgctaaagttgaccagctgagcaacgacgtgaacgcaatgcgttccgacgttcaggctgctaaagatgacgcagctcgtgctaaccagcgtctggacaacatggctactaaataccgcaagtaa

SEQ ID NO: 2 - protein, 78 aa, Escherichia coli
  SIGNAL (1)..(20): Lpp signal peptide
MKATKLVLGAVILGSTLLAGCSSNAKIDQLSSDVQTLNAKVDQLSNDVNAMRSDVQAAKDDAARANQRLDNMATKYRK

SEQ ID NO: 3 - DNA, 90 bp, Klebsiella pneumoniae
  misc_feature: CGTase signal sequence
atgaaaagaaaccgtttttttaatacctcggctgctattgccatttcgattgcattaaatacttttttttgtagcatgcagacgattgct

SEQ ID NO: 4 - DNA, 70 bp, Artificial Sequence
  Oligonucleotide lpp1
cagcgttcgatgcttctttgagcgaacgatcaaaaataagtgccttcccacatatgaatatcctccttag

SEQ ID NO: 5 - DNA, 70 bp, Artificial Sequence
  Oligonucleotide lpp2
gcgcacaatgtgcgccatttttcacttcacaggtactattacttgcggtagtgtaggctggagctgcttc

SEQ ID NO: 6 - DNA, 23 bp, Artificial Sequence
  Oligonucleotide pykF
ctcgcgcagtacgtaaatacttc

SEQ ID NO: 7 - DNA, 21 bp, Artificial Sequence
  Oligonucleotide ynhG2
cgtctttgcttacactcacgc

SEQ ID NO: 8 - DNA, 452 bp, Artificial Sequence
  DNA molecule comprising the lpp1 allele
cgggatccatgaaagctactaaactggtactgggcgcggtaatcctgggttctactctgctggcaggttgctccagcaacgctaaaatcgatcagctgtcttctgacgttcagactctgaacgctaaagttgaccagctgagcaacgacgtgaacgcaatgcgttccgacgttcaggctgctaaagatgacgcagctcgtgctaaccagcgtctggacaacatggctactaaatactgcaagtaatagtacctgtgaagtgaaaaatggcgcacattgtgcgccattttttttgtctgccgtttaccgctactgcgtcacgcgtaacatattcccttgctctggttcaccattctgcgctgactctactgaaggcgcattgctggctgcgggagttgctccactgctcaccgaaaccggataccctgcccgacgatacaacgctggatcccg

SEQ ID NO: 9 - DNA, 455 bp, Artificial Sequence
  DNA molecule comprising the lpp3 allele
cgggatcctctcactttatctaagatgaatccgatggaagcatcctgttttctctcaatttttttatctaaaacccagcgttcgatgcttctttgagcgaacgatcaaaaataagtgccttcccatcaaaaaaatattctcaacataaaaaactttgtgtaatacttgtaacgctacatggagattaactcaatctagagggtattaataatgaaagctactaaactggtactgggcgcggtaatcctggattctactctgctggcaggttgctccagcaacgctaaaatcgatcagctgtcttctgacgttcagactctgaacgctaaagttgaccagctgagcaacgacgtgaacgcaatgcgttccgacgttcaggctgctaaagatgacgcagctcgtgctaaccagcgtctggacaacatggctactaaataccgcaagtaaggatcccg

SEQ ID NO: 10 - DNA, 2065 bp, Klebsiella pneumoniae
  misc_feature: DNA molecule comprising the CGTase gene
ataatgaaaagaaaccgtttttttaatacctcggctgctattgccatttcgattgcattaaatacttttttttgtagcatgcagacgattgctgctgaaccagaagaaacttatcttgattttcgtaaggagacgatatattttctattccttgatcgtttcagcgatggagatccaagtaataatgcagggtttaattctgcaacctacgatcctaataatttaaaaaaatatactggaggagatctccgggggttgattaataaactaccctatttaaaatcacttggtgttacttcaatctggattactcccccaatcgataatgtgaataatactgatgctgctggcaatactggatatcatggttattggggaagagattattttcgtatagatgaacattttggcaatctcgatgatttcaaagaactgactagtttgatgcatagtcctgattataatatgaaactggttcttgattatgcccctaatcattcgaatgctaatgatgaaaatgaatttggtgcactatatcgtgatggtgtgtttattactgattatcctacgaatgttgccgccaatacgggctggtatcatcacaatggtggggtaacgaactggaatgatttcttccaagtgaagaatcataatctattcaatctatcagacctcaatcaatccaatactgatgtctaccagtacttgttggatggttctaaattttggatcgatgctggtgtggatgctatcaggattgatgccatcaagcatatggacaagtcttttatacagaaatggaccagcgatatttatgattacagtaagtctatcggccgggaaggattttttttcttcggtgaatggtttggtgccagtgcgaatactacaacaggtgttgatggtaatgctatcgattacgccaacacttccgggtcagcgttgctggattttggattccgcgatactttagaaagagttttggtaggacgtagcggaaatacaatgaaaacgttaaatagttatctgataaaaagacaaacagtctttaccagtgatgactggcaggttgtttttatggataaccatgatatggcacgcattggtaccgctctgcgttcaaacgccactacttttggtcctggaaataatgaaaccggtggaagtcagagtgaagcttttgctcagaaacgtatagacctcggtctggttgcgacaatgactgtacgtggtattcctgccatttattatggtactgaacattatgccgctaactttacctctaacagttttggtcaagttggcagtgatccttacaaccgagagaaaatgccaggatttgatacggaaagtgaggctttctccattattaaaacactgggtgacctaaggaaaagtagcccggcaattcaaaatggaacttatactgaactatgggttaatgatgatatattagtatttgagcggcgttctgggaacgatattgttattgttgcacttaatcgtggtgaggctaacacaattaatgttaaaaatatagcggttcctaatggggtatatccgagtttgattgggaataatagtgtttcagtagcaaataaacggacaacactaacacttatgcaaaatgaagctgttgtcattcgctcacaatcagatgatgcggagaaccctacagtacaaagcataaacttcacatgtaataacggttatacgatttcaggtcaaagtgtttatattattggtaatatacctcagttaggtggttgggacttaactaaagcggtaaaaatatcaccgacacaatatccacaatggagtgcgagcttagagcttccttctgacttaaatgttgaatggaagtgtgtgaaacgtaatgaaaccaatccgacggctaatgttgagtggcagtctggtgcaaataaccagttcaatagcaatgacacacaaacaacgaatggctcgttttaattaaaatttagtggaccagcgttccaatcgatggtccactattcgtactccggccataattatttttgactaatactcttacaaattttcaacc

SEQ ID NO: 11 - DNA, 652 bp, Artificial Sequence
  DNA molecule comprising the interferon alpha2b gene
cagaattctaaggaggaaattatatgaaaagaaaccgtttttttaatacctcggctgctattgccatttcgattgcattaaatacttttttttgtagcatgcagacgattgcttgtgacttacctcagacccattcactgggctcacgccgtacgctgatgctgttagcacagatgcgtcgcatttctctgtttagttgtttgaaagaccgtcatgattttgggttcccgcaagaagagtttggtaatcagtttcagaaagccgaaactattccggttctgcacgaaatgattcaacagatttttaacctgttttcgacaaaggatagctctgccgcgtgggatgaaaccttactggataagttctacaccgaactgtaccagcaactgaatgatctggaagcatgcgttatccagggcgtgggtgtcacagaaactccgctgatgaaggaggacagcattctggcggtgcgcaaatatttccagcgtatcacgctgtatctgaaagagaaaaaatattcgccatgcgcgtgggaggtcgtgcgcgcggagatcatgcgcagtttctctttgagcaccaacctgcaagaatccttgcgttccaaagaataatagtctagaagcttggctgttttggcggatgagctgcagaa

SEQ ID NO: 12 - DNA, 769 bp, Artificial Sequence
  DNA molecule comprising the gene of the heavy chain  of the anti-lysozyme Fab fragment
cagaattctaaggaggaaattatatgaaaaagacagctatcgcgattgcagtggcactggctggtttcgctaccgtagcgcaggctgaagttaaactgcaagaatccggtccgggtctggttgctccgtcccagtccctgtccatcacctgcaccgtttccggtttctccctgaccggttacggtgttaactgggttcgtcagccgccgggtaaaggtctggaatggctgggtatgatctggggtgacggtaacaccgactacaactccgctctgaaatcccgtctgtccatctccaaagacaactccaaatcccaggttttcctgaaaatgaactccctgcacaccgacgacaccgctcgttactactgcgctcgtgaacgtgactaccgtctggactactggggtcagggtaccaccgttaccgtttcctccgctaaaaccaccccgccgtccgtttacccgctggctccgggttccgctgctcagaccaactctatggttaccctgggttgcctggttaaaggttacttcccggaaccggttaccgttacctggaactccggttccctgtcctccggttgccacaccttcccggctgttctgcaatccgacctgtacaccctgtcctcctccgttaccgttccgtcctccacctggccgtccgaaaccgttacctgcaacgttgctcacccggcttcctccaccaaagttgacaaaaaaatcgttccgcgtgaccatcaccaccatcaccattaataactgcagaa

SEQ ID NO: 13 - DNA, 768 bp, Artificial Sequence
  DNA molecule comprising the gene of the light chain of the anti-lysozyme Fab fragment
aactgcagtacatggagaaaataaaatgaaaagaaaccgtttttttaatacctcggctgctattgccatttcgattgcattaaatacttttttttgtagcatgcagacgattgctgacatcgaactgacccagtccccggcttccctgtccgcttccgttggtgaaaccgttaccatcacctgccgtgcttccggtaacatccacaactacctggcttggtaccagcagaaacagggtaaatccccgcagctgctggtttactacaccaccaccctggctgacggtgttccgtcccgtttctccggttccggttccggtacccagtactccctgaaaatcaactccctgcaaccggaagacttcggttcctactactgccagcacttctggtccaccccgcgtaccttcggtggtggtaccaaactggaactgaaacgtgctgacgctgctccgaccgtttccatcttcccgccgtcctccgaacagctgacctccggtggtgcttccgttgtttgcttcctgaacaacttctacccgaaagacatcaacgttaaatggaaaatcgacggttccgaacgtcagaacggtgttctgaactcctggaccgaccaggactccaaagactccacctactccatgtcctccaccctgaccctgaccaaagacgaatacgaacgtcacaactcctacacctgcgaagctacccacaaaacctccacctccccgatcgttaaatccttcaaccgtaacgaataatagctgcagaa

SEQ ID NO: 14 - DNA, 1441 bp, Artificial Sequence
  DNA molecule comprising the gene for the heavy chain of the anti-TF antibody
cagaattctaaggaggaaattatatgaaaaagacagctatcgcgattgcagtggcactggctggtttcgctaccgtagcgcaggctgaggttcagctggtggagtctggcggtggcctggtgcagccagggggctcactccgtttgtcctgtgcagcttctggcttcaatattaaggagtactacatgcactgggtccgtcaggccccgggtaagggcctggaatgggttggattgattgatccagagcaaggcaacacgatctatgacccgaagttccaggaccgtgccactataagcgctgacaattccaaaaacacagcatacctgcaaatgaacagcctgcgtgctgaggacactgccgtctattattgtgctcgagacacggccgcttacttcgactactggggtcaaggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataataactgcagaa

SEQ ID NO: 15 - DNA, 771 bp, Artificial Sequence
  DNA molecule comprising the gene for the light chain of the anti-TF antibody
aactgcagtacatggagaaaataaaatgaaaagaaaccgtttttttaatacctcggctgctattgccatttcgattgcattaaatacttttttttgtagcatgcagacgattgctgatatccagatgacccagtccccgagctccctgtccgcctctgtgggcgatagggtcaccatcacgtgcagagccagtcgcgacatcaagagctatctgaactggtatcaacagaaaccaggaaaagctccgaaagtactgatttactatgctactagtctcgctgaaggagtcccttctcgcttctctggatccggttctgggacggattacactctgaccatcagcagtctgcaaccagaagacttcgcaacttattactgtcttcagcacggagagtctccgtggacatttggacagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatagctgcagaa